(19) United States
(12) Patent Application Publication
(10) Pub. No.: US 2005/0222085 A1
(43) Pub. Date: Oct. 6, 2005

(54) Quaternised ammonium cyclodextrin compounds

(76) Inventors: Georg Ludwig Kis, Triboltingen (CH); Christian Schoch, Muttenz (CH); Jozsef Szejtli, Budapest (HU)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/516,247

(22) PCT Filed: Jun. 12, 2003
(86) PCT No.: PCT/EP03/06192

§ 371 Date: Nov. 30, 2004

(30) Foreign Application Priority Data
Jun. 13, 2002 (EP) 02013074.6
Dec. 20, 2002 (EP) 02028554.0

(51) Int. Cl. A61K 31/724; C08B 37/16

(52) U.S. Cl. 514/58; 536/46

(57) ABSTRACT

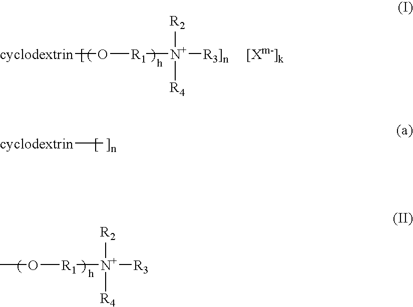

Use of a compound of formula (I), wherein the symbol (a) represents a n-valent residue derived from a cyclodextrin compound by removing n of its hydroxyl groups; n is a number greater than 0 and represents the average number of substituents of formula (II) per molecule of said compound; h is 0 or 1; R₁ is a di-valent group selected from alkylene, hydroxy alkylene, halogeno alkylene, monocyclic aralkylene, cycloalkylene and phenylene; R₂, R₃ and R₄ are each independently of one another a group selected from alkyl, cycloalkyl, aryl, aralkyl and cycloheteryl; X^{m-} is a m-fold negatively charged anion; m is an integer being equal or greater than 1; and k is n/m in the preparation of an anti-infective medicament, as preservative and penetration enhancer.

The present invention relates to novel uses of quaternized ammonium cyclodextrin compounds (hereinafter also QACD compounds) having the formula (I): 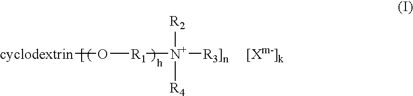
and to novel pharmaceutical, particularly ophthalmic compositions comprising said compounds and their uses.

The compounds of formula (1), wherein
cyclodextrin [symbol] ]ₙ
represents a n-valent residue derived from a cyclodextrin (=CD) compound by removing n of its hydroxyl groups;
n is a number greater than 0 and represents the average number of substituents of formula 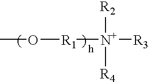
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
X^{m-} is a m-fold negatively charged anion;
m is an integer being equal or greater than 1; and
k is n/m,
   are known and described, for instance, in U.S. Pat. No. 3,453,257 (index h=1) or U.S. Pat. No. 5,241,059 (index h=0).

The compounds of formula (I) are described in said references as being better soluble in water than non-cationic cyclodextrin compounds like, for example, beta-cyclodextrin itself. Based on these findings, U.S. Pat. No. 5,241,059 states in very general terms that compounds of formula (I) may be useful for making pharmaceuticals, for example. In a more recent investigation (T. Loftsson et al., Drug Development and Industrial Pharmacy, 24(4), 365-370 (1998)) It has furthermore been found that cationic cyclodextrin derivatives like e.g. 2-hydroxy-3-trimethyl-ammoniopropyl-beta-cyclodextrin, have a reduced solubilizing effect on organic compounds in water in comparison to uncharged cyclodextrins, like 2-hydroxypropyl-beta-cyclodextrin or randomly methylated beta-cyclodextrin, in particular on zwitterionic compounds, especially on the anti-inflammatory drug ETH-615 having the following formula:

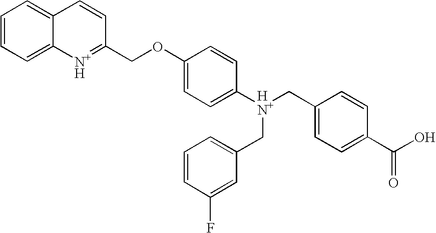

In a first aspect, the present invention is based on the surprising findings that the compounds of formula (I) are potent anti-microbial agents, in particular effective against bacteria and fungi. Furthermore, said compounds are also useful against viruses. The compounds of formula (I) exhibit a very low toxicity as shown, for example, by a LD₅₀ in mice of 750-1500 mg/kg) and exhibit excellent tolerability. Based on their anti-microbial activity the compounds of formula (I) can be used, on one side, as anti-infective drugs for the treatment of infective diseases caused by the presence of bacteria, fungi or viruses and, on the other side, as potent preservatives for all types of compositions which require preservation, in particular pharmaceutical compositions.

Therefore, in a first aspect the instant invention relates to the use of compounds of the aforementioned formula (I) in the preparation of an anti-infective medicament, that means a medicament for the treatment of bacterial, fungal and viral infections, in particular bacterial and fungal infections of a mammal, particularly a human, in need of such treatment.

The compounds of formula (I) represent a new class of anti-infective agents, and may therefore be particular valuable in view of controlling infections of microorganisms, which are resistant against one or more of anti-infective agents presently in use, like antibiotics. Furthermore the new compounds may provide a further alternative for treating patients who cannot tolerate one or more known ant-infective compounds or are allergic against the known compounds.

The compounds of formula (I) are substances derived from the well-known cyclodextrins, a group of homologous oligosaccharides. As also well known, cyclodextrins are homologous cyclic molecules containing 6 or more, especially 6, 7 or 8 alfa-D-glucopyranose units linked together at the 1,4 positions as in amylose. When the number of alfa-D-glucopyranose units is 6, the molecule is known as an alfa-cyclodextrin, when the number of alfa-D-gluco-pyranose units is 7, the molecule is known as a beta-cyclodextrin; and when this number is 8, the molecule is known as gamma-cyclodextrin. For the purposes of this application the term “cyclodextrin” is intended to include the aforementioned forms, as well as other corresponding cyclic molecules that have a still larger number of alfa-D-glucopyranose units in the molecule, and, as well, mixtures of these and, optionally, other homologs. The various homologous cyclodextrins, having from six to eight units, or more, and their mixtures, may be used as equivalent materials for the purposes of this invention. In practice, there may be little reason for separating the various fractions, and the cyclodextrin employed may contain a preponderance of specific cyclodextrin, e.g. beta-cyclodextrin. No distinction is intended between the various homologous cyclodextrins or their mixtures unless otherwise indicated, when using the term “cyclodextrin”.

The term quaternized ammonium cyclodextrin compound of the instant invention also includes the corresponding derivatives wherein one or more of the free hydroxyl groups have been etherified, in particular corresponding methyl ether derivatives.

Preferred cyclodextrin compounds according to the invention include the compounds of formula (Ia): 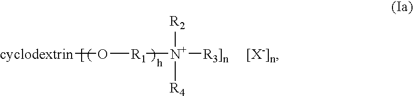
wherein X⁻ is a simple-charged anion and the other residues and h and n have the meaning as in formula (I).

In formula (I) and (Ia) R₁ may be an alkylene, a hydroxy alkylene, an otherwise substituted alkylene, an aralkylene, a cycloalkylene, or a phenylene radical. Thus, R₁ may be, for instance, a branched or straight chain C₁-C₈alkylene, for Instance methylene, ethylene, a propylene, butylene, pentylene, hexylene or an octylene group etc. The foregoing and other alkylene radicals also may be substituted in one or more places, for instance, by a hydroxyl, alkoxy, aryl or cycloalkylene radical derived from, for example, cyclopropane, cyclobutane and higher homologues. R₁ may also represent a phenylene radical which may be substituted, if desired e.g. by alkoxy, alkyl, preferably C₁-C₄alkyl or halogen.

R₂, R₃ and R₄ may be different or the same, and may be alkyl, aryl, aralkyl, cycloalkyl, cycloheteryl.

Thus, R₂, R₃ and R₄ may, for instance, be C₁-C₁₈alkyl, like methyl, ethyl, n-propyl, i-propyl, n-butyl, sec-butyl, t-butyl, a branched or straight chain pentyl, hexyl, heptyl, octyl, decyl or octadecyl and the like alkyl radicals, and may also be substituted by one or more substituents, preferably one or more hydroxyl or halogen substituent. Preferably alkyl means C₁-C₁₀alkyl, for instance C₁-C₈alkyl, especially C₁-C₄alkyl, very specially methyl.

In the meaning cycloalkyl R₂, R₃ and R₄ may, preferably, represent C₃-C₆cycloalkyl, like for example, cyclopropyl, cyclobutyl, cyclopentyl or cyclohexyl, which may also be substituted by one or more substiuents, e.g. by C₁-C₄alkyl, hydroxyl or halogen.

As aryl group R₂, R₃ and R₄, are preferably phenyl which may be substituted, for instance by alkyl, in particular C₁-C₄alkyl, or halogen.

In the meaning cycloheteryl R₂, R₃ and R₄ represent the residue of a heterocyclic group, for instance morpholinyl, pyridyl, pyrrolidyl, furfuryl, imidazolidyl, imidazolyl and the like.

X^{m-} is a m-fold negatively charged anion, preferably a halide, including bromide, chloride and iodide, nitrate, phosphate, sulfate, formate, acetate, butyrate, oleate, stearate, benzoate anion, or the like, whereas [X⁻] is a simple-charged anion, preferably also one selected from those above.

In preferred compounds of formula (I) or (Ia) R₂, R₃ and R₄ represent same or different C₁-C₁₈alkyl radicals. Specific preference is thereby given to those alkyl radicals already mentioned above.

As is known, each cyclodextrin molecule of a compound of formula (I) or (Ia) may have a different degree of substitution. The total number of quaternized ammonium groups In the molecule corresponding to the Index n, may range from 1 to the maximum number of hydroxyl groups of the base cyclodextrin, that means theoretically up to 18 in case of alfa-cyclodextrin, 21 in case of beta-cyclodextrin and 24 in case of gamma-cyclodextrin. In a given quantity of a cyclodextrin derivative, there will however generally be some cyclodextrin molecules that are not substituted at all, together with other molecules that have different numbers of quaternized ammonium substituents. A statistical average is therefore employed to characterize the number of quaternized ammonium groups of the entire quantity of cyclodextrin (molecules). The present invention embraces QACD compounds of formula (I) or (Ia) having an index n ranging from a value slightly above 0, for instance from about 0.1 to the theoretical upper values indicated above. This necessarily implies that the QACD derivatives may be used in the form of a mixture with other materials, such as unreacted cyclodextrin, and, as well, in substantially pure form. According to the art, the primary 6 position hydroxyl groups in any anhydroglucose group appear to be the most reactive, followed by the hydroxyl groups at the 2-position which are believed to be the next most reactive, and the hydroxyl at the 3-position as the least reactive. Irrespective of the actual sequence or order of reactions or the number of anhydroglucose units involved, the formulae (I) and (Ia) are intended to represent the QACD derivatives wherein the quaternized ammonium substitution may occur in different degrees of substitution at all or less than all anhydroglucose units in the cyclodextrin. Preferably n ranges from about 1 to 8, more preferably from about 1 to 4, for example from 1 to 3.

The QACD derivatives of formula (I) or (Ia), wherein the index h is 1, may, for instance, be prepared as described in detail in U.S. Pat. No. 3,453.257 or analogically. The compounds of formulae (I) and (Ia), wherein the index h is 0, may, for example, be prepared as described in U.S. Pat. No. 5,241,059.

Specific embodiments of compounds useful for the instant invention include the compounds of formula (Ia), wherein h is 1 and wherein R₁ is a branched or straight chain C₁-C₈alkylene or phenylene, which both may be substituted In one or more places; R₂, R₃ and R₄ are different or the same, and represent substituted or unsubstituted C₁-C₁₈alkyl; C₃-C₆cycloalkyl; phenyl; morpholinyl, pyridyl, pyrrolidyl, furfuryl, imidazolidyl or imidazolyl and X⁻ is a halide, nitrate, formate, acetate, butyrate, oleate, stearate, benzoate anion.

Particularly preferred is the use of compounds of formula (Ia), wherein R₁ is a branched or straight chain C₁-C₈alkylene, in particular C₁-C₄alkylene, more preferably ethylene or propylene, and R₂, R₃ and R₄ are different or the same, and are substituted or unsubstituted C₁-C₁₈alkyl, more particularly C₁-C₈alkyl, specifically C₁-C₄alkyl.

Preferred as well Is the use of the compounds of formula (I) or (Ia), wherein the symbol
cyclodextrin [symbol] ]ₙ
represents a n-valent residue of alfa-, beta- or gamma-cyclodextrin including the corresponding partially or fully etherified compounds.

The instant invention furthermore relates to an ant-infective pharmaceutical composition comprising an anti-microbially active drug selected from the compounds of formula (I) and particularly of formula (Ia) as described above.

Such pharmaceutical compositions according to the invention are suitable for enteral, such as oral or rectal, parenteral, such as by intravenous, subcutaneous, intramuscular, or transdermal administration to mammals including humans, and useful for the treatment of infective disorders responsive thereto and comprise an anti-microbially effective amount of a QACD compound of formula (I) or preferably of formula (Ia) as the pharmacologically active compound, alone or in combination, with one or more pharmaceutically acceptable carriers and/or excipients suitable for either enteral or parenteral application.

Such compositions may, for instance, be in the form of tablets or gelatin capsules and comprise, for instance, one or more of the QACD compounds of formula (I) or (Ia) as active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbants, colorants, flavors and sweeteners. Injectable compositions according to the invention are preferably aqueous isotonic solutions or suspensions, and suppositories are advantageously prepared from fatty emulsions or suspensions. Said compositions may be sterilized, if desired, and contain adjuvants, such as stabilizing, wetting or emulsifying agents, salts for regulating the osmotic pressure and/or buffers. If desired, such compositions may also comprise additional preserving and/or solubilizing agents, although this is usually not necessary. Suitable formulations for transdermal application include an effective amount of one or more QACD compound of formula (I) or (Ia) with a carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Other forms of pharmaceutical compositions according to the invention include inhalation formulations, emulsions, suspensions, rods, inserts, implants.

Suitable formulations for topical application, especially but not restricted to the skin and the eyes, include aqueous solutions, ointments, creams or gels well-known in the art. Compositions according to the invention for topical use are particularly advantageous because the QACD compounds of formula (I) and (Ia) have unexpectedly been found to exhibit, in addition to their anti-microbial effect, firstly an excellent topical tolerability and secondly a particularly improved permeability through tissue like skin and especially ocular tissue, in particular corneal and/or conjunctival tissue. Especially advantageous embodiments of the compositions according to the invention are therefore ophthalmic compositions comprising one or more compound of formula (I) or (Ia). Said compositions according to the Invention may also contain other therapeutically valuable compounds and are generally prepared according to conventional mixing, granulating or coating methods, respectively, and contain about 0.1 to 75%, preferably about 1 to 50%, of the active ingredient.

In a further aspect the instant Invention relates to preserved pharmaceutical compositions comprising a preservative selected from the compounds of formula (I), and in particular from compounds of formula (Ia) and additionally one or more pharmaceutically active, preferably ophthalmic drug other than a compound of said formula (I) and (Ia). Such compositions comprise the compounds of formula (I) or (Ia) in preservatively effective amounts, for instance 0.01 to 10% based on the total weight of the composition, preferably 0.1 to 10%, e.g. 0.1 to 5%. If desired, said compositions may also comprise conventional preservatives in addition to the compounds of formulae (I) and (Ia), like for instance other quaternary ammonium compounds such as benzalkonium chloride (N-benzyl-N—(C₈-C₁₈alkyl)-N,N-dimethylammonium chloride), benzoxonium chloride or preservatives different from quaternary ammonium salts like alkyl-mercury salts of thiosalicylic acid, such as, for example, thiomersal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid, in particular if the preservative efficacy of such combinations is increased when compared to the same amount of only one of the preservatives. A further specific embodiment of compositions according to the instant invention is free of such conventional preservatives.

A further unexpected advantage of the QACD compounds of formula (I) and (Ia) is their compatibility with aqueous solutions of hyaluronic acid compounds, in particular hyaluronic acid salts, e.g. sodium hyaluronate, which are a valuable and frequently used formulation component of pharmaceutical, in particular of ophthalmic compositions, however, are known to be incompatible with benzalkonium chloride, a very common preservative especially of ophthalmic compositions, together with which they irreversibly form a precipitate. The QACD compounds of formula (I) or formula (Ia) present an elegant way out of this dilemma because they provide an excellent preservative efficiacy like benzalkonium chloride in combination with a good compatibility with hyaluronic acid derivatives which are Incompatible with benzalkonium chloride.

As already mentioned above, the QACD compounds of formula (I) and (Ia) have furthermore proved to enhance the permeability of human or animal tissue, in particular of corresponding ocular and mucus tissue, for other drugs, specifically for drugs which form an inclusion complex with the respective QACD compounds. In a further aspect the present Invention therefore relates to the use of compounds of formula (I) or (Ia) for enhancing the permeation of a drug through tissue and for enhancing the penetration of a drug into tissue, wherein said drug is preferably a drug typically administered topical to said tissue, and, in still a further aspect, to compositions comprising one or more pharmaceutical drug other than a compound of formula (I) or (Ia) and an enhancer for the permeability of said drug through or into the human or animal tissue, in particular ocular or mucus tissue, which enhancer is selected from the compounds of formula (I) and the compounds of formula (Ia) as described above.

A specific embodiment of said compositions does not comprise a zwitterionic drug in general, and/or specifically the drug ETH-615. described above.

Suitable drugs include but are not limited to anti-angiogenic drugs, anti-inflammatory drugs, anti-allergic drugs, anesthetic drugs, myopia preventing/inhibiting drugs, miotics, carbonic anhydrase inhibitors, alpha blocking agents, antioxidants, vitamins, and biologic materials like peptides, proteins, DNAS, RNAs and the like substances.

Specifically suitable drugs include furthermore ophthalmic and/or ocularly tolerable, which may, for example, be selected from the following drugs and combinations thereof:
anti-inflammatory drugs, such as steroids, e.g. dexamethasone, fluorometholone, hydrocortisone, prednisolone; or so-called non-steroidal anti-inflammatory drugs (NSAID) such as COX-inhibitors, e.g. diclofenac, ketorolac, or indomethacin;
anti-allergic drugs, selected e.g. from FK506, 33-epi-chloro-33-desoxy-ascomycin, cromolyn, emadine, ketotifen, levocabastine, iodoxamide, norketotifen, olopatadine, and rizabene;
drugs to treat glaucoma (in particular intraocular pressure treatment), selected e.g. from latanoprost, 15-keto-latanoprost, unoprostone isopropyl, betaxolol, clonidine, levobunolol and timolol;
anesthetic drugs, e.g. selected from cocaine hydrochloride, lidocaine, oxybuprocaine and tetracaine hydrochloride;
myopia preventing/inhibiting drugs such as pirenzepine, atropine and the like;
miotics, e.g. selected from carbachol, pilocarpine and physostigmine;
carbonic anhydrase inhibitors, e.g. selected from acetazolamide and dorzolamide;
alpha blocking agents, e.g. selected from apraclonidine and brimonidine; and
antioxidants and/or vitamins, e.g. selected from ascorbic acid, retinol, retinol acetate, retinol palmitate, and natural and synthetic tocopherols, in particular alfa-tocopherol and alfa tocopherol acetate; and
biologic materials like peptides, proteins, DNAs, RNAs and the like substances, and if desired from
antifungal drugs, e.g. selected from amphotericin B, fluconazole and natamycin; and
anti-viral drugs such as acyclovir, fomivirsen, ganciclovir, and trifluridine.

The addressed drugs are specifically useful for the topical treatment of a disease other than an infective disease, a fungus related disease, a viral disease, for example for the treatment of glaucoma, inflammation, allergy (such as hay fever), analgesia (e.g. surgery, mechanical ocular impacts, etc.) and the like. Particularly preferred ophthalmic drugs are therefore selected from anti-inflammatory drugs, anti-allergic drugs, and drugs to treat glaucoma.

It is known to those skilled in the art that a specific cyclodextrin compound does not automatically form an inclusion complex with any randomly chosen other compound, which may be desired. In such cases it is therefore preferred to use a QACD derivate derived from a cyclodextrin compound, for instance alfa-, beta- or gamma-cyclodextrin, that meets the cavity needs of the other component or components, e.g. the pharmaceutical drug/drugs.

The amount of compounds of formula (I) or formula (Ia) present in such an ophthalmic composition generally depends on the ophthalmic drug being used is typically in the range of 0.01-35%, preferably from 0.5-25%, e.g. from 5-10%, 10-15% and 15-20%. Also preferred are amount from 0.1-5% of the compound of formula (I) or (Ia), in particular 0.5-5% and most particular 1-5% of said compound, by total weight of a corresponding ophthalmic composition.

The compositions for topical administration and/or ophthalmic compositions comprise advantageously a carrier suitable for topical administration, such as for example water, mixtures of water and water-miscible solvents, such as C₁-C₇-alkanols, vegetable oils or mineral oils comprising from 0.05 to 10%, preferably 0.5 to 5% by weight hydroxyethylcellulose, ethyl oleate, carboxymethylcellulose, and other non-toxic water-soluble polymers for ophthalmic uses, such as, for example, cellulose derivatives, such as methylcellulose, alkali metal salts of carboxymethylcellulose, hydroxymethylcellulose, methylhydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof. Highly preferred is water. The concentration of the carrier is, for example, from 1 to 100000 times the concentration of the active ingredient.

The ophthalmic compositions of the present invention may further comprise a tonicity enhancing agent. Tonicity enhancing agents are, for example, ionic compounds, such as boric acid, or alkali metal or alkaline earth metal halides, such as, for example, CaCl₂, KBr, KCl, LiCl, Nal, NaBr or NaCl. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol, preferred from 100 to 400 mOsmol, more preferred from 200 to 400 mOsmol and even more preferred from 280 to 350 mOsmol.

For the adjustment of the pH, preferably to a physiological pH, buffers may especially be useful. Examples of buffer substances are acetate, ascorbate, borate, hydrogen carbonate/carbonate, citrate, gluconate, lactate, phosphate, propionate and TRIS (tromethamine) buffers. Tromethamine and borate buffer are preferred buffers. The amount of buffer substance added is, typically, that necessary to ensure and maintain a physiologically tolerable pH range. The pH range is generally in the range of from 4 to 9, preferably from 4.5 to 8.5 and more preferably from 5.0 to 8.2.

Even though the compositions of the present Invention may further comprise an additional preservative, e.g on storage or to inhibit microbial growth after opening a closed container holding such a composition and exposing such a composition to the air, this is normally not necessary because of the ant-microbial effect of the QACD compounds of formula (I) and (Ia). The term “anti-microbial effect” is meant to include an anti-bacterial, anti-fungal and anti-viral effect, in particular an anti-bacterial and an anti-fungal effect.

A composition of the present invention may additionally require the presence of a solubilizer, in particular if the active or the inactive ingredients tend to form a suspension or an emulsion.

A solubilizer suitable for compositions of the invention may, for example, be selected from the group consisting of tyloxapol, fatty acid glycerol polyethylene glycol esters, fatty acid polyethylene glycol esters, polyethylene glycols, glycerol ethers, other cyclodextrin compounds (for example alpha-, beta- or gamma-cyclodextrin, e.g. alkylated, hydroxyalkylated, carboxyalkylated or alkyloxycarbonyl-alkylated derivatives, or mono- or diglycosyl-alfa-, -beta- or gamma-cyclodextrin, mono- or dimaltosyl-alfa-, -beta- or -gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH 40®. Reaction products of castor oil and ethylene oxide appear to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is typically from 0.1 to 5000 times the concentration of the active Ingredient.

A composition of the invention may comprise further non-toxic excipients, such as, for example, emulsifiers, wetting agents or fillers, such as, for example, polyethylene glycols, e.g. having an average molecular weight of 200, 300, 400 and 600, or higher polyethylene glycols, such as Carbowax 1000, 1500, 4000, 6000 and 10000. Other excipients that may be used if desired are listed below but are not intended to limit in any way the scope of the possible excipients. They are especially complexing agents, such as disodium-EDTA or EDTA, antioxidants, such as ascorbic acid, acetylcysteine, cysteine, sodium hydrogen sulfite, butyl-hydroxyanisole, butyl-hydroxytoluene or alpha-tocopherol acetate; stabilizers, such thiourea, thiosorbitol, sodium dioctyl sulfosuccinate or monothioglycerol; or other excipients, such as, for example, lauric acid sorbitol ester, triethanol amine oleate or palmitic acid ester. Preferred exipients are complexing agents, such as disodium-EDTA. The amount and type of excipient added is in accordance with the particular requirements and is generally in the range of from approximately 0.0001 to approximately 90% by weight.

A further problem frequently arises when a drug is topically administered, namely that it is often washed off again from the issue to which it is applied during an unacceptably short time period. Compositions comprising one or more pharmaceutical drug, a compound of formula (I) or (Ia) as described hereinabove and a carrier comprising a polymer surprisingly provide a solution to this problem, too. It has namely been found that said compositions allow a sustained or prolonged drug delivery at the place of administration.

Therefore the present invention includes a further aspect, the use of a composition comprising one or more pharmaceutical drug, a compound of formula (I) or (Ia) and a carrier comprising a polymer as a drug dosage system for sustained delivery.

Compositions according to the instant invention which are especially suitable as a drug dosage system for sustained delivery are preferably in a semi-solid (paste-like) or more preferably a solid state and are, for example, in the form of a film, a rod, a bar, a capsule, a corneal shield a corneal ring, an implant, an insert, an intraocular lens, a therapeutic contact lens, a tablet, e.g. a mini tablet, a mini-disc, or a pellet.

Carriers suitable for solid state medicaments representing a dosage system for sustained drug delivery according to the invention are for example selected from
a matrix of a bioerodible polymer preferably being selected from the group consisting of polyhydroxy-acids, such as polylactic acid and polyglycolic acid; polyesters, polyorthoesters, polyanhydrides, polycyanoacrylates, natural gums, such as acacia gum and arabic gum; celluloses, such as carboxymethylcellulose; methacrylate (co)polymers such as Eudragits, e.g. Eudragit RL PO, Eudragit RS PO; and/or
a bioadhesive polymer preferably being selected from the group consisting of maltodextrin, celluloses, such as carboxymethyl cellulose, hydroxyethyl cellulose; chitosans; hyaluronic acid; polyacrylates e.g. carbopol; polycarbophils e.g. Noveon AA-1; polyvinylalcohol such as Mowiol 2688; polyvinylpyrrolidone such as povidone K30.

Accordingly, the invention relates to a solid state medicament selected from a film, a rod, a bar, a capsule, a corneal shield a corneal ring, an Implant, an insert, an intra-ocular lens, a therapeutic contact lens, a tablet, e.g. a mini tablet, a mini-disc, and a pellet comprising a pharmaceutically effective drug, a compound of formula (I) or (Ia) and a carrier suitable for the manufacure of a solid state medicament. Said solid state medicaments provide in particular the synergistic advantage of sustained drug delivery with improved drug permeability.

The physical properties/appearances of a solid state medicament according to the invention depends on the carriers used, and its physical appearance may, for example, range from soft to stiff, from water soluble up to water insoluble, from transparent to opaque and so on.

The concentration of an above carrier is, for example, from 1 to 100000 times the concentration of the active ingredient.

An even more specific aspect of the instant invention is an ophthalmic composition comprising a QACD compound of formula (I) or (Ia), at least a further pharmaceutically active component like an ophthalmic drug which is a novel drug delivery system providing the synergistic properties of improved drug delivery, improved tolerability and excellent preservative efficacy. Drug delivery as used herein refers particularly to topical ocular administration.

Still further aspects of the instant invention are
a method for preserving a pharmaceutical composition comprising adding to said pharmaceutical composition an effective amount of a preservative selected from the compounds of formula (I); preferably selected from the compounds of formula (Ia);
a method for enhancing the permeability of a drug contained in a pharmaceutical composition through skin, buccal, mucosal, pulmonal, vaginal or ocular, in particular conjunctival tissue, more particularly for enhancing the permeability of a drug contained in an ophthalmic composition through ocular tissue, especially the corneal permeation, comprising adding to said composition a compound selected from the compounds of formula (I), preferably selected from the compounds of formula (Ia);
a method for synergistically enhancing the permeability of a drug contained in a pharmaceutical composition through skin, buccal, mucosal, pulmonal, vaginal or ocular, in particular conjunctival tissue, more particularly for enhancing the permeability of a drug contained in an ophthalmic composition through ocular tissue, especially the corneal permeation, and for preserving said composition comprising adding to said composition a compound selected from the compounds of formula (I), preferably selected from the compounds of formula (Ia); and
a method for controlled, in particular, sustained or prolonged delivery of an organic compound, in particular a pharmaceutically active drug at the place of administration, comprising the following steps:
   mixing said drug at least with a compound selected from the compounds of formula (I) corresponding to claim 1, in particular selected from the compounds of formula (Ia) according to claim 2 and a polymer, in particular one or more of a bioerodible polymer and/or one or more of a bioadhesive polymer, and administering the composition obtained thereby.

### EXAMPLE 1

### Increase of Corneal Permeation

Corneal Permeation Device:

The device used is a modified Valia-Chien system consisting of two water-jacketed cells for temperature control. Each cell is filled with GBR buffer (see below), stirred by a magnet and continuously gassed with Oxycarbon (5% CO₂/95% O₂). During an experiment, the cells are separated by the cornea, one cell containing the test substance dissolved in GBR and acting as donor (tear side), the other one being the acceptor (aqueous humor side).

Corneas:

Pig eyes are obtained from the local abattoir. They are kept in Dulbecco's MEM (minimal essential medium) with Glutamax-I (Gibco) on ice and used within a few hours after receipt.

Buffers:

Buffers for in vitro corneal permeation studies are adapted from glutathione-bicarbonate-Ringer (GBR) solution. “GBR aqueous humor” is used in the acceptor cell and “GBR tears” on the donor side for equilibration. Their composition is listed in Table 1.

Assay of Corneal Permeation:

On receipt from the abattoir the eye is mounted on a dissection board, cornea facing up. After checking integrity of the cornea, the sclera is incised approximately 1-2 mm from the corneal rim with a scalpel and the anterior segment is excised. The iris and lens are carefully removed with forceps without damaging the corneal structures. The cornea is then mounted between the two cells of the permeation device with the help of a pinch clamp. Immediately, 3 ml of previously warmed and gassed GBR buffer are added to each cell, carefully removing any trapped air bubbles in the cells. The system is gassed and stirred for about 30 minutes at 35° C. After equilibration, the donor side is emptied and the same amount of a previously warmed formulation of active substance is added at time t=0. An aliquot of 300 μl “GBR aqueous humor” is taken at time t=0 from the acceptor cell and the missing volume is replaced by the same volume of fresh buffer. Subsequently, this procedure is repeated in the acceptor cell at predefined time points and the aliquots are analysed for active by HPLC. Both compartments are kept under constant stirring with small magnets. The usual duration of an experiment is 180 minutes which is also the time of contact of the formulation with the cornea.

TABLE 1
Buffers used for in vitro corneal permeation experiments

| | GBR aqueous humor | GBR tears |
|---|---|---|
| Constituent | Concentration [mM] | Concentration [mM] |
| NaCl | 95.75 | 115.75 |
| NaH₂PO₄ | 1.25 | 1.25 |
| KCl | 4 | 20 |
| CaCl₂ | 2 | 2 |
| MgCl₂ | 1 | 1 |
| Adenosine | 0.5 | 0.5 |
| NaHCO₃ | 23 | 23 |
| Glutathione reduced | 0.3 | 0.3 |
| Glucose | 77.7 | 27.75 |
| H₂O | q.s. | q.s. |
| pH | 7.3-7.4* | 7.3-7.4* |
| Osmolality | 297 mOsm/kg | 311 mOsm/kg |

*when gassed with 5% CO₂/95% O₂

A) Corneal permeation experiments with diclofenac formulations

1) Diclofenac sodium 0.1% without thiomersal (marketed Voltaren Ophtha formulation, SDU)

| | Average permeated amount | |
|---|---|---|
| Time (min) | (micro-gram) | S.D. |
| 0 | 0 | 0 |
| 30 | 0 | 0 |
| 60 | 0 | 0 |
| 90 | 0.1970182 | 0.160121 |
| 120 | 0.5716975 | 0.385907 |
| 180 | 1.6826328 | 0.78374 |

2) Diclofenac sodium 0.1% with 2% HP-gamma-cyclodextrin and without BAC

| | Average permeated amount | |
|---|---|---|
| Time (min) | (micro-gram) | S.D. |
| 0 | 0 | 0 |
| 30 | 0 | 0 |
| 60 | 0.2512672 | 0.237461 |
| 90 | 1.2153835 | 0.532895 |
| 120 | 2.2474393 | 0.707873 |
| 180 | 6.4313489 | 1.643572 |

3) Diclofenac sodium 0.1% with 0.1% OA-beta-CD and without BAC

| | Average permeated amount | |
|---|---|---|
| Time (min) | (micro-gram) | S.D. |
| 0 | 0 | 0 |
| 30 | 0.263057 | 0.15532 |
| 60 | 1.767697 | 1.093765 |
| 90 | 5.807919 | 1.235891 |
| 120 | 10.71147 | 2.608262 |
| 180 | 20.08034 | 3.651529 |

BAC=benzalkonium chloride
HP-gamma-cyclodextrin=hydroxypropyl-gamma-cyclodextrin
QA-beta-CD=3-(trimethylammonio)-2-hydroxypropyl-beta-cyclodextrin chloride

In the above experiments the efficacy in drug permeation an embodiment of this invention (QA-beta-CD [item 3]) can directly be compared with respect to the prior art situation (HP-gamma-cyclodextrin; [item 2)]).

B) Corneal permeation experiments with 5-methyl-2-(2′ chloro-6′-fluoroanilino)phenyl acetic acid (Compound B)

1) Compound B 0.1% without thiomersal (analogous to marketed Voltaren Ophtha formulation, SDU)

| | Average permeated amount | |
|---|---|---|
| Time (min) | (micro-gram) | S.D. |
| 0 | 0 | 0 |
| 30 | 0 | 0 |
| 60 | 0.0840 | 0.1361 |
| 90 | 0.4359 | 0.2176 |
| 120 | 0.8800 | 0.3568 |
| 180 | 2.0137 | 0.4391 |

2) Compound B 0.1% with 2% HP-gamma-cyclodextrin and without BAC

| | Average permeated amount | |
|---|---|---|
| Time (min) | (micro-gram) | S.D. |
| 0 | 0 | 0 |
| 30 | 0 | 0 |
| 60 | 0.9456 | 0.7494 |
| 90 | 3.6156 | 2.1221 |
| 120 | 5.5475 | 2.8703 |
| 180 | 10.4593 | 4.4961 |

3) Compound B 0.1% with 0.1% QA-beta-cyclodextrin and without BAC

| | Average permeated amount | |
|---|---|---|
| Time (min) | (micro-gram) | S.D. |
| 0 | 0 | 0 |
| 30 | 0 | 0 |
| 60 | 2.2883 | 1.4595 |
| 90 | 6.7793 | 2.8701 |
| 120 | 12.4568 | 2.7525 |
| 180 | 21.9144 | 5.1823 |

BAC=benzalkonium chloride
HP-gamma-cyclodextrin=hydroxypropyl-gamma-cyclodextrin
QA-beta-cyclodextrin=3-(trimethylammonio)-2-hydroxypropyl-beta-cyclodextrin chloride, empiric formula: (C₆H₁₀₋ₙO₅)₇(C₆ H₁₅ONCl)ₙ=2-5.

Again the efficacy in drug permeation of an embodiment of this invention (QA-beta-CD [item 3)]) can directly be compared with respect to the prior art situation (HP-gamma-cyclodextrin; [item 2)]), in these experiments.

### EXAMPLE 2

### Preservative Effectiveness Test

0.2 g of QA-beta-CD (QA-beta-CD=3-(trimethylammonio)-2-hydroxypropyl-beta-cyclodextrin chloride, empiric formula: (C₆H₁₀₋ₙO₅)₇(C₆H₁₅ONCl)ₙ n=2-5. are added to 170 ml water (nanopure). Then 10.10 g of Sorbitol and 0.2 g of Disodium edetate are added and the pH is adjusted to a value of 7.0-7.4 and water added to make up 200 ml of resulting solution. The solution is filtered through a Corning bottle top filter unit 0.22 μm CA under sterile conditions. The solution has a pH of 7.35 and an osmolality of 307 mOsm/kg (270-330).

Samples of the solution are inoculated with Escherichia coli ATCC 8739; Pseudomonas aeruginosa ATCC 9027, Staphyloccocus aureus ATCC 6538; Candida albicans ATCC 10231 and Aspergillus niger ATCC 16404, respectively and assayed for presence/growth at the times given in Table 2.

TABLE 2

| | Escherichia | Pseudomonas | Staphyloccocus | Candida | Aspergillus |
|---|---|---|---|---|---|
| | coli | aeruginosa | aureus | albicans | niger ATCC |
| | ATCC 8739 | ATCC 9027 | ATCC 6538 | ATCC 10231 | 16404 |
| Inoculum | 2.5 10⁶ | 2.0 10⁶ | 1.1 10⁶ | 1.1 10⁶ | 2.9 10⁵ |
| Time 0 h | 2.5 10⁵ | 2.2 10⁵ | 1.5 10⁵ | 1.4 10⁵ | 1.1 10⁴ |
| Time 6 h | <10² | <10² | 5.4 10³ | — | — |
| Time 24 h | <10² | <10² | <10² | — | — |
| Time 7 d | <10² | <10² | <10² | <10² | 2.7 10³ |
| Time 14 d | — | — | — | <10² | 2 10² |
| Time 21 d | — | — | — | — | — |
| Time 28 d | <10² | <10² | <10² | <10² | 2. 10² |

The tested solution meets the requirements as defined as European Pharmacopeia (Eur. Ph.) criteria A for ophthalmic preparations, e.g. as described in Eur. Ph. Supplement 2001, Section 5.1.3, page 293 to 295.

### EXAMPLE 3

Films able to act as ocular Insert are prepared and tested. The compositions of the films are disclosed in Table 3.

All preparations are made under magnetic stirring (400 rpm) and at room temperature. Firstly, the glycerol is dissolved In 5 ml of double distilled water or, alternatively, In 5 ml of aqueous phosphate buffer (pH=7) for the composition A and B. Then the Mowiol 26-88 is added to said water or said phosphate buffer comprising said glycerol until complete dissolution. The D-mannitol or QA-beta-CD are dissolved in the resulting solution and after complete dissolution of these components, the polyvinylpyrrolidone is added to the mixture. At last, ketotifen hydrogenfumarate is added under stirring, and the resulting solutions are then centrifuged (500×g, 25 min, 25° C.) in order to remove impurities. All solutions were very clear.

The solutions are then cast in petri dishes and dried in a desiccator (containing phosphor pentoxyde) under vacuum (13 mbar). Upon drying, the resulting thin layer films are scored according the semi quantitative scale of Table 4. Two Individuals conducted said evaluation blindly and independently.

Table 5 shows the mean value of the overall score (possible range 0 to 9) of the thin layer films cast in the Petri dishes.

After drying, the preparations comprising Mowiol 26-88, glycerol and QA-beta-CD give excellent results. The maximum score is obtained for the basic preparation loaded with QA-beta-CD (E). This score is significantly higher than the one obtained when the preparation is loaded with D-mannitol (B). The resulting thin layer film is highly transparent, easy to remove from the cast support and it exhibits a regular aspect and is essentially homogeneous. The addition of polyvinylpyrrolidone in the preparation is efficient and the resulting films exhibit good scores. This is particularly the case when the preparations are loaded with QA-beta-CD (D).

Furthermore the compositions F to K are prepared, analogous to the compositions A and D but loaded with different amounts of ketotifen hydrogen fumarate (Zaditen), namely 6.75, 12.5 and 20% respectively. The resulting layers evidence good and equivalent scores with 6.75 and 12.5% of drug (F and I and G and J respectively). When the films are loaded with 40 mg (20%) of Zaditen (H, K), the presence of some crystalline structures is found in the film based on D-mannitol. This re-crystallization does not occur In presence of QA-beta-CD, probably because of a certain Interaction between the QACD component and the drug.

Appropriate thin layer films are obtained with the addressed compositions containing QA-beta-CD.

TABLE 3
Thin layer film composition (in mg. per 200 mg film)

| Composition | A | B | C | D | E | F | G | H | I | J | K |
|---|---|---|---|---|---|---|---|---|---|---|---|
| Mowiol 26-88 | 100 | 140 | 100 | 100 | 140 | 100 | 100 | 100 | 100 | 100 | 100 |
| Hydroxypropyl cellulose | | | 40 | | | | | | | | |
| Polyvinylpyrrolidone | 40 | — | | 40 | — | 40 | 40 | 40 | 40 | 40 | 40 |
| D-mannitol | 50 | 50 | | | | 36.5 | 25 | 10 | | | |
| QA-beta-CD | | | 50 | 50 | 50 | | | | 36.5 | 25 | 10 |
| Glycerin | 10 | 10 | 10 | 10 | 10 | 10 | 10 | 10 | 10 | 10 | 10 |
| Ketotifen hydrogen fumarate | | | | | | 13.5 | 25 | 40 | 13.5 | 25 | 40 |

TABLE 4
Semi quantitative scale used to score the thin layer films

| Criterion | Observation | Score |
|---|---|---|
| Thin layer film formed | Yes | 1 |
| | No | 0 |
| Transparency | Good | 3 |
| | Average | 2 |
| | Poor | 1 |
| | None | 0 |
| Surface aspect | Regular | 1 |
| | Non-regular | 0 |
| Homogeneity | Yes | 1 |
| | No | 0 |
| Remove from cast support | Easy | 3 |
| | Average | 2 |
| | Difficult | 1 |
| | None | 0 |

TABLE 5

| Composition | Macroscopic Score |
|---|---|
| A | 7.5 |
| B | 7.5 |
| C | 5.5 |
| D | 8 |
| E | 9 |
| F | 7.5 |
| G | 8 |
| H | 8 |
| I | 7.5 |
| J | 8.5 |
| K | 9 |

1-33. (canceled)

34. An anti-infective pharmaceutical composition comprising an antimicrobially active drug of formula (I) 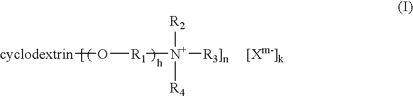
wherein the symbol
cyclodextrin [symbol] ]ₙ
represents a n-valent residue derived from a cyclodextrin compound by removing n of its hydroxyl groups;
n is a number greater than 0 and represents the average number of substituents of formula 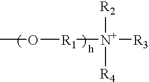
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
X^{m-} is a m-fold negatively charged anion;
m is an integer being equal or greater than 1; and
k is n/m.

35. The anti-infective composition according to claim 34, wherein the drug of formula (I) is a compound of formula (Ia)

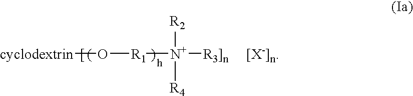

36. The anti-infective composition according to claim 34, wherein the composition is topically administered to the skin or eye in the form of an aqueous solution, ointment, cream or gel.

37. A pharmaceutical composition comprising a preservative, wherein the preservative is a compound of formula (I) 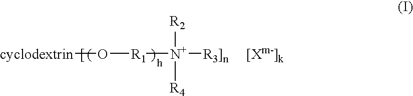
wherein the symbol
cyclodextrin [symbol] ]ₙ
represents a n-valent residue derived from a cyclodextrin compound by removing n of its hydroxyl groups;
n is a number greater than 0 and represents the average number of substituents of formula 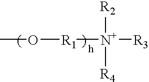
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
Xₘ₋ is a m-fold negatively charged anion;
m is an integer being equal or greater than 1; and
k is n/m; and
additionally one or more pharmaceutically active drugs other than a compound of formula (I).

38. The pharmaceutical composition according to claim 37, wherein the preservative of formula (I) is a compound of formula (Ia)

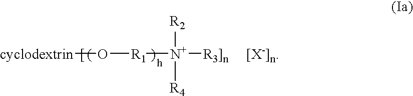

39. The pharmaceutical composition according to claim 37, wherein the one or more pharmaceutically active drugs is an ophthalmic drug.

40. The pharmaceutical composition according to claim 37, wherein the preservative is present in a concentration of 0.01 to 10% by weight of the total composition.

41. An ophthalmic composition comprising one or more ophthalmic drugs and an enhancer for the permeability of said drug through ocular tissue, wherein the drug is a compound of formula (I) 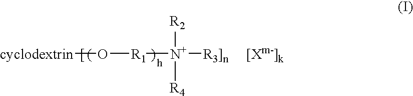
wherein the symbol
cyclodextrin [symbol] ]ₙ
represents a n-valent residue derived from a cyclodextrin compound by removing n of its hydroxyl groups;
n is a number greater than 0 and represents the average number of substituents of formula 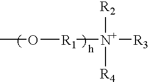
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
X^{m-} is a m-fold negatively charged anion;
m is an integer being equal or greater than 1; and
k is n/m.

42. The ophthalmic composition of claim 41, wherein the compound of formula (I) is a compound of formula (Ia)

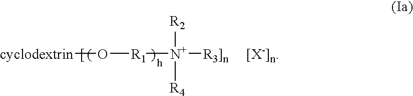

43. The ophthalmic composition according to claim 41, wherein the ocular tissue is a corneal tissue.

44. The ophthalmic composition according to claim 41, wherein the enhancer is present in a concentration ranging from 0.01-35%.

45. The ophthalmic composition according to claim 41, wherein the one or more ophthalmic drugs is selected from the group consisting of anti-inflammatory drugs, anti-allergic drugs, drugs to treat glaucoma, anesthetic drugs, myopia preventing/inhibiting drugs, miotics, carbonic anhydrase inhibitors, alpha blocking agents, antioxidants, vitamins and biologic materials.

46. The ophthalmic composition according to claim 45, wherein the ophthalmic drug is selected from anti-inflammatory drugs, anti-allergic drugs, and drugs to treat glaucoma.

47. The ophthalmic composition according to claim 41, further comprising one or more ophthalmically acceptable excipients.

48. The ophthalmic composition according to claim 47, wherein the excipients comprise a salt of hyaluronic acid.

49. The ophthalmic composition according to claim 41, which is substantially free of benzalkonium chloride.

50. A drug dosage system for sustained delivery consisting essentially of a composition comprising one or more pharmaceutically active drugs, a compound of formula (I) 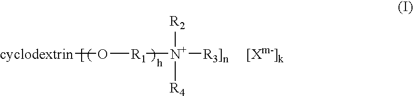
wherein the symbol
cyclodextrin [symbol] ]ₙ
represents a n-valent residue derived from a cyclodextrin compound by removing n of its hydroxyl groups;
n is a number greater than 0 and represents the average number of substituents of formula 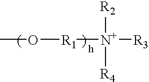
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
X^{m-} is a m-fold negatively charged anion;
m is an integer being equal or greater than 1; and
k is n/m, and a carrier comprising a polymer and selected from a film, a rod, a bar, a capsule, a corneal shield. a corneal ring, an implant, an insert, an intra-ocular lens, a therapeutic contact lens, a tablet, mini tablet, a mini-disc, and a pellet.

51. A method for preserving a pharmaceutical composition, the method comprising:
adding to said pharmaceutical composition an effective amount of a preservative, wherein the preservative is a compound of formula (I) 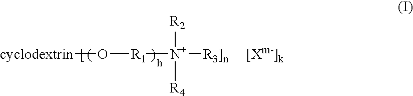
wherein the symbol
cyclodextrin [symbol] ]ₙ
represents a n-valent residue derived from a cyclodextrin compound by removing n of its hydroxyl groups;
n is a number greater than 0 and represents the average number of substituents of formula 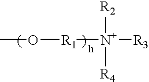
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
X^{m-} is a m-fold negatively charged anion;
m is an integer being equal or greater than 1; and
k is n/m.

52. The method of claim 51, wherein the compound of formula (I) is a compound of formula (Ia)

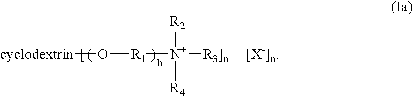

53. A method for enhancing the permeability of a drug contained in a pharmaceutical composition through skin, buccal, mucosal, pulmonal, vaginal or ocular tissue, the method comprising:
adding to said composition a compound of formula (I) 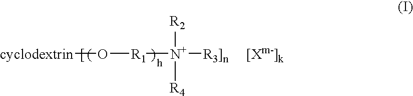
wherein the symbol
cyclodextrin [symbol] ]ₙ
represents a n-valent residue derived from a cyclodextrin compound by removing n of its hydroxyl groups;
n is a number greater than 0 and represents the average number of substituents of formula 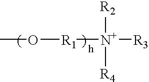
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
X^{m-} is a m-fold negatively charged anion;
m is an integer being equal or greater than 1; and
k is n/m.

54. The method according to claim 53, wherein the compound of formula (I) is a compound of formula (Ia)

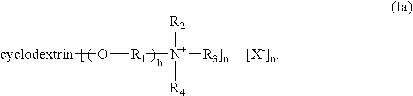

55. The method according to claim 53, wherein the ocular tissue is conjunctival or corneal tissue.

56. A method for sustained or prolonged delivery of a pharmaceutically active drug at the place of administration, comprising the following steps:
mixing said drug at least with a compound of formula (I) 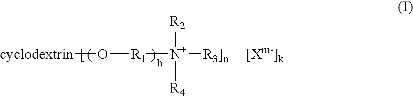
wherein the symbol
cyclodextrin [symbol] ]ₙ
represents a n-valent residue derived from a cyclodextrin compound by removing n of its hydroxyl groups;
n is a number greater than 0 and represents the average number of substituents of formula 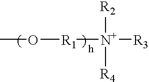
per molecule of said compound;
h is 0 or 1;
R₁ is a di-valent group selected from alkylene, hydroxy alkylene, halogeno alkylene, monocyclic aralkylene, cycloalkylene and phenylene;
R₂, R₃ and R₄ are each independently of one another a group selected from alkyl, cycloalkyl, aryl, aralkyl, and cycloheteryl;
X^{m-} is a m-fold negatively charged anion;
m is an integer being equal or greater than 1; and
k is n/m.
and a polymer; and
administering the composition obtained thereby.

57. The method of claim 56, wherein the compound of formula (I) is a compound of formula (Ia) [chemical structure]
